(19) United States
(12) Patent Application Publication
(10) Pub. No.: US 2016/0135782 A1
(43) Pub. Date: May 19, 2016

(54) FINGER JOINT ULTRASOUND IMAGING

(71) Applicant: General Electric Company, Schenectady, NY (US)

(72) Inventors: Dongqing Chen, New Berlin, WI (US); Xiaodong Han, Shanghai (CN); Gang Cheng, Shanghai (CN); Menachem Halmann, Bayside, WI (US); Kun Tao, Shanghai (CN); Zhenyu Liu, Wuxi (CN); Hong Wang, Shanghai (CN)

(21) Appl. No.: 14/541,827

(22) Filed: Nov. 14, 2014

(51) Int. Cl. A61B 8/08; A61B 5/00; A61B 8/00

(52) U.S. Cl. CPC A61B 8/0875; A61B 8/40; A61B 8/4281; A61B 8/4483; A61B 8/463; A61B 8/488; A61B 8/5246; A61B 8/54; A61B 5/0082; A61B 5/4528

(57) ABSTRACT

A method of acquiring ultrasound images for a rheumatoid arthritis examination comprises receiving a hand in a scanning assembly including a transducer array and a fluid providing an acoustic coupling between the transducer array and the hand and identifying locations of a plurality of finger joints of the hand while the hand is held stationary in the scanning assembly. Ultrasound images of the plurality of finger joints are acquired with the transducer array while the hand is held stationary in the scanning assembly, wherein the ultrasound images are of an area less than an entire area of the hand based on the identified locations of the finger joints of the hand.

### BACKGROUND

In many circumstances, it is beneficial to obtain ultrasound images of finger joints such as when diagnosing hand injuries or when such joints become inflicted with a disease such as rheumatoid arthritis. With current methods and equipment, it is often difficult to capture images of such finger joints in a consistent and efficient manner.

### BRIEF DESCRIPTION OF THE DRAWINGS

FIG. 1 is a schematic diagram of an example finger joint ultrasound imaging system.

FIG. 2 is a flow diagram of an example method for acquiring ultrasound images of a finger joint.

FIG. 3 is a flow diagram of another example method for acquiring ultrasound images of a finger joint.

FIG. 4 is a schematic diagram of another example finger joint ultrasound imaging system.

FIG. 5 is a flow diagram of another example method for acquiring ultrasound images of a finger joint.

FIG. 6 is a perspective view of another example finger joint ultrasound imaging system.

FIG. 7 is an enlarged fragmentary view of the finger joint ultrasound imaging system of FIG. 6 receiving a hand.

FIG. 8 is another enlarged fragmentary view of the finger joint ultrasound imaging system of FIG. 6 receiving the hand.

FIG. 9 is a schematic diagram of another example finger joint ultrasound imaging system.

FIG. 10 is a flow diagram of an example method for acquiring ultrasound images on a finger joint.

FIG. 11 is a screenshot of an example display presented by the system of FIG. 9.

### DETAILED DESCRIPTION OF EXAMPLES

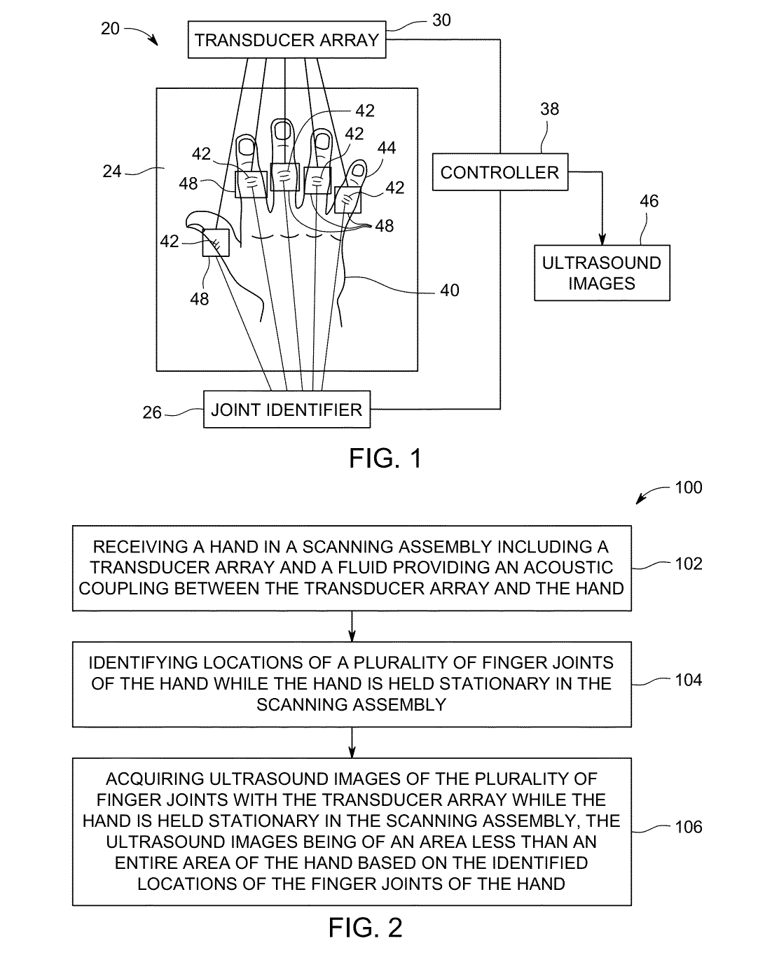

FIG. 1 schematically illustrates an example finger joint ultrasound imaging system 20. As described hereafter, finger joint ultrasound imaging system 20 consistently captures images of finger joints in an efficient manner. In the example illustrated, finger joint ultrasound imaging system 20 comprises an ultrasound scanning assembly comprising fluid 24, transducer array 30, joint location identifier 34 and controller 38.

Fluid 24 comprise a volume of fluid which serves as an acoustic coupling between a person's hand 40 and transducer array 30. In one implementation, fluid 24 comprises a bath of water in which hand 40 is immersed during scanning by transducer array 30 of imaging system 20. In other implementations, fluid 24 may comprise other forms of a fluid, such as other forms of a liquid, gel or the like, which serve as an acoustic coupling between hand 40 and transducer array 30.

Transducer array 30 comprises an array of transducers that output signals to facilitate the acquisition of ultrasound images of the joints of hand 40. In the example illustrated, transducer array 30 comprises quartz crystals, piezoelectric crystals, that change shape in response to the application electrical current so as to produce vibrations or sound waves. Likewise, the impact of sound or pressure waves upon such crystals produce electrical currents. As a result, such crystals are used to send and receive sound waves. Each of the transducers of transducer array 30 may additionally include a sound absorbing substance to eliminate back reflections and an acoustic lens to focus emitted sound waves.

Joint location identifier 34 comprises a device by which locations of a joint or multiple joints of hand 40 are provided to controller 38. In the example illustrated, joint location identifier 34 identifies the location of the joints 42, which are the second knuckles of each of the fingers, digits or phalanges 44. For purposes of this disclosure, the term “fingers” includes a person's thumb as well as the remaining digits of a hand. In other implementations, joint location identifier 34 identifies other joints of hand 40. As will be described hereafter, the identified locations of joints 42 are used by controller to control the operation and/or positioning of transducer array 30.

In one implementation, joint location identifier 34 determines the location of phalange or finger joints 42 based simply upon a predefined positioning of hand 40 when in imaging system 20. For example, in one implementation, imaging system 20 may include hand fixtures or other structures which retain hand 40 against movement in a predefined geo-referenced position and orientation. As a result, the positioning of joints 42 is largely consistent from one imaging session to another imaging session or from one person to another person. Based upon this predefined position and orientation of hand 40, as defined by such hand fixtures, joint location identifier 34, embodied as programming or code and an associated processor, identifies or estimates the location of finger joints 42.

In another implementation, joint location identifier 34 comprises a device which senses positioning of hand 40, whereby the location of joints 42 are derived from the sense positioning of hand 40. In one implementation, joint location identifier 34 comprises one or more sensors which are contacted by portions of hand 40 when hand 40 is held stationary within fluid 24, wherein joint location identifier 34 comprises programming or software and a processor that utilizes signals from such sensors to determine the positioning of hand 40 and derive the location of joints 42 from the positioning of hand 40. In another implementation, joint location identifier 34 comprises one or more optical cameras and associated image recognition programming or software and a processor, whereby the location of joints 42 are derived from a determined positioning of hand 40 or whereby location of joints 42 are directly determined from signals received from the one or more optical cameras. In yet another implementation, joint location identifier 34 utilizes signals from transducer array 30 itself, wherein a processor, under the direction of programming or software, derives location of joints 42 from the detected positioning of hand 40 or directly determines location of joints 42 using such signals from transducer array 30. In yet other implementations, other sensing devices or imaging devices are utilized to sense the positioning of hand 40 from which the location of joints 42 are derived or to directly sense the location of joints 42.

In yet other implementations, joint location identifier 34 comprises an input device, such as a keyboard, mouse or the like, by which a person manually inputs and provides controller 38 with manually identified locations of joints 42.

Controller 38 comprises a processor or processing unit that receives the locations of joints 42 and that utilizes such signals to control the operation and/or positioning of transducer array 30. For purposes of this disclosure, the term “processing unit” shall mean a presently developed or future developed processing unit that executes sequences of instructions contained in a memory. Execution of the sequences of instructions causes the processing unit to perform steps such as generating control signals. The instructions may be loaded in a random access memory (RAM) for execution by the processing unit from a read only memory (ROM), a mass storage device, or some other non-transitory persistent storage. In other embodiments, hard wired circuitry may be used in place of or in combination with software instructions to implement the functions described. For example, controller 38 may be embodied as part of one or more application-specific integrated circuits (ASICs). Unless otherwise specifically noted, the controller is not limited to any specific combination of hardware circuitry and software, nor to any particular source for the instructions executed by the processing unit.

Controller 38 controls the operation and/or positioning of transducer array 30 so as to generate joint focused ultrasound images 46 of focus regions 48, which are less than the entire area of hand 40. Each focus region 48 serves as a window from which the joint focused images of the finger joints are taken. In one implementation, controller 38 controls the operation and/or positioning of transducer array 30 such that ultrasound pulses are directed at and/or from just those focus regions 48, wherein ultrasound pulses are not directed at regions outside of focus regions 48 and/or are not received from regions of hand 40 outside of focus regions 48. In another implementation, controller 38 controls the operation and/or positioning of transducer array 30 such that transducer array 30 directs ultrasound pulses at and receives ultrasound pulses with respect to portions of hand 40 larger than focus regions 48, but where transducer array 30 operates differently with respect to focus regions 48 as compared to portions of hand 40 outside of focus regions 48. For example, in one implementation, controller 38 controls transducer array 30 such that a higher density, closer spacing or greater frequency of ultrasound pulses is directed at and received from focus regions 48 as compared to portions of hand 40 outside of focus regions 48. In one implementation, each of focus regions 48 has an area less than or equal to 9 square centimeters. In one implementation of each of focus regions 48 has a width of less than or equal to 3 cm.

In one implementation, controller 38 adjusts the positioning of transducer array 30 based upon identified locations of joints 42. For example, in one implementation, controller 38 outputs control signals which cause an actuator to move transducer array 30 so as to focus ultrasound imaging on the focus regions 48 extending about the identified locations of joints 42. In one implementation in which signals from transducer array 30 are also used by joint location identifier 34 to identify the locations of joints 42, controller 38 locates transducer array 30 farther away from hand 40 to sense all of hand 40 or portions of hand 40 so as to encompass multiple joints 42 during the location of joints 42. Once the locations of joints 42 have been determined, controller 38 uses such determined locations to generate or establish a focus region 48 about each of joints 42, wherein the controller 38 outputs control signal to locate transducer array 30 in closer proximity to hand 40 and to move transducer array 30 between different positions in close proximity to each of the focus regions 48. For example, in one implementation, once the location of joints 42 have been determined and focus regions 48 have been generated for each of the determined locations of joints 42, controller 38 generates control signals directing an actuator to move transducer array 30 at first locating speed into close proximity with hand 40 and adjacent to focus region 48 over joint 42 of the person's thumb. In one implementation, controller 38 generates control signals directing the actuator to move transducer array 30 at a slower second imaging speed to scan across focus region 48 over joint 42 of the person's thumb. Once ultrasound images are acquired for focus region 48 across joint 42 of the thumb, controller 38 generates control signals directing the actuator to move transducer array 30 at the first higher locating speed to a location adjacent the next focus region to be imaged. This process is repeated until joint focused ultrasound images 46 have been completed for each of the joints 42 to be imaged. The acquired joint focused ultrasound images 46 are stored in memory. In one implementation, the acquired joint focus ultrasound images 46 are further displayed.

Because controller 38 utilizes the determined locations of joints 42 to form focus regions 48 and because controller 38 focuses transducer array 30 on just those focus regions 48, rather than the entire hand 40, imaging time is reduced and efficiency is increased. As a result, additional time may be spent on such focus regions 48 to increase the amount of imaging data acquired for joints 42. In implementations where controller 38 repositions transducer array 30 at each of the focus regions 48 by moving transducer array 30 at different speeds, a first greater speed when moving between focus regions 48 and a second slower speed when scanning across each focus region 48, imaging time is reduced and efficiency is increased.

Although the example illustrated in FIG. 1 illustrates each focus region 48 as being square in shape, in other implementations, each focus region 48 may have other shapes, such as a circular shape, and oval shape or the like. In one implementation, the shape of each focus region 48 corresponds to a general outline of the area of hand 48 constituting joint 42. Although the example of FIG. 1 illustrates each focus region 48 as having the same size across each of joints 42 of hand 40, in other implementations, focus regions 48 have different sizes and/or different shapes amongst the different joints 42 of hand 40. For example, in one implementation, the size and/or shape of a particular focus region 48 varies depending upon which phalange joint 42 is being imaged. In one implementation, the size and/or shape of each focus region 48 varies based upon a determined level of confidence regarding the location of the particular joint 42 as determined by joint location identifier 34. For example, the determined or estimated location of a first joint 42 may have a higher level of precision or expected accuracy as compared to the estimated location of the second joint 42. In such a circumstance, controller 38 generates or utilize a larger focus region 48 for the second joint 42 as compared to the size of the focus region utilized for the first joint 42 to provide the focus region 48 for the second joint 42 with a greater tolerance for joint location estimation variability and potential inaccuracy.

FIG. 2 is a flow diagram of an example method 100 for imaging finger joints 42 of hand 40. In one implementation, method 100 may be carried out by system 20 of FIG. 1. In other implementations, method 100 may be carried out by other imaging systems.

As indicated by block 102 in FIG. 2, method 100 comprises the step of receiving a hand, such as hand 40, in a scanning assembly, such a imaging system 20, including a transducer array 30 and a fluid 24 providing an acoustic coupling between the transducer array 30 and the hand 40. In one implementation, the hand 40 is simply lowered into a basin or tank containing the fluid 24. In another implementation, the hand 40 is inserted through a hole in the basin or tank containing fluid 24.

As indicated by block 104, method 100 further comprises a step of identifying locations of a plurality of finger joints 42, of the hand 40 while the hand is held stationary in the imaging system 20. In one implementation, imaging system 20 comprises various indentations, projections or other structures that guide the positioning of hand 40 within imaging system 20 and which also restrain movement of hand 40 while within imaging system 20 such that the positioning of hand 40 is uniform or consistent from one imaging session to another imaging session and from one person to another person. In one implementation, imaging system 20 comprises structures that position and retain each individual finger of hand 40. In one implementation, imaging system 20 comprises a series of upwardly extending posts which guide positioning of the fingers of hand 40, which separate the fingers of hand 40, and which restrain movement of the fingers of hand 40.

In one implementation, the location of finger joints 42 is estimated or identified by joint location identifier 34. As noted above, in one implementation, the location of joints 42 is estimated identified using one or more sensors which are contacted by portions of hand 40 when hand 40 is held stationary within fluid 24. In another implementation, the location of joints 42 are derived from a determined positioning of hand 40 or whereby location of joints 42 are directly determined from signals received from the one or more optical cameras. In yet another implementation, the locations of joints 42 are derived from the detected positioning of hand 40 or are determined directly using such signals from transducer array 30. In yet other implementations, other sensing devices or imaging devices are utilized to sense the positioning of hand 40 from which the location of joints 42 are derived or to directly sense the location of joints 42. Still other implementations, the locations of finger joints 42 are determined and manually input to controller 38.

As indicated by block 106 of FIG. 2, method 100 comprises the step of acquiring ultrasound images of the plurality of finger joints with the transducer array 30 while the hand 40 is held stationary in the imaging system 20, wherein the joint focused images 46 are of an area less than the entire area of hand 40 based upon the identified locations of the finger joints 42 of hand 40. In one implementation, controller 38 controls the operation and/or positioning of transducer array 30 based upon the identified locations of finger joints 42 of hand 40. In one implementation, controller 38 generates a focus region 48 based upon and for each of the identified locations of joints 42, wherein imaging is focused on the focus regions 48. As a result, imaging efficiency is enhanced.

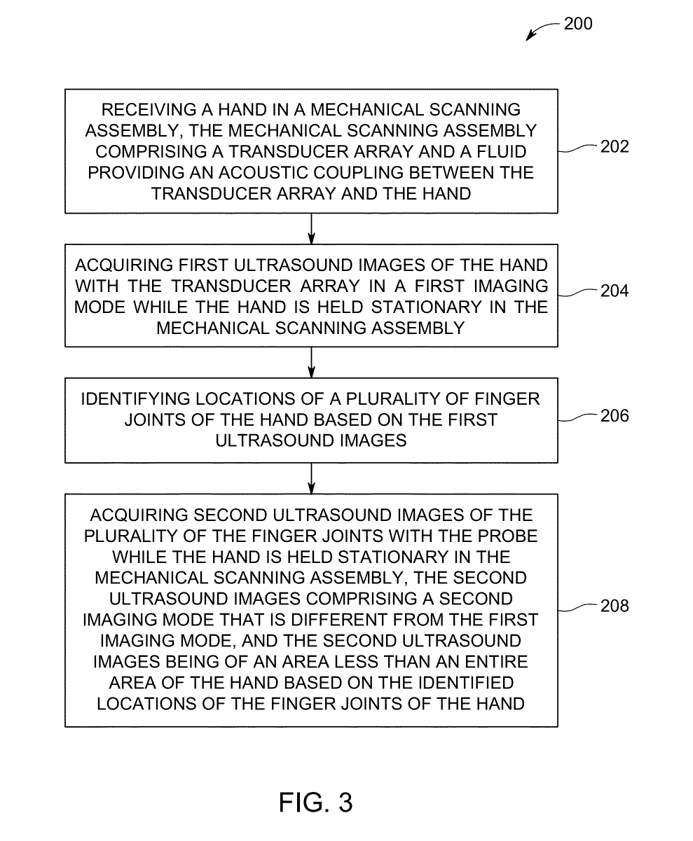

FIG. 3 is a flow diagram of another example method 200 for imaging finger joints. In one implementation, method 200 may be carried out by system 20. In another implementation, method 200 may be carried out by other ultrasound imaging systems.

As indicated by block 202 of FIG. 3, method 200 comprises receiving a hand 40 in a mechanical scanning assembly 20, wherein the mechanical scanning assembly, such as system 20, comprises a transducer array 30 in a fluid 24 providing an acoustic coupling between the transducer array 30 and the hand 40.

As indicated by block 204 of FIG. 3, method 200 comprises acquiring first ultrasound images of the hand 40 with the transducer array 30 in a first imaging mode while the hand 40 is held stationary in the mechanical scanning assembly. As noted above, in one implementation, imaging system 20 comprises various indentations, projections or other structures that guide the positioning of hand 40 within imaging system 20 and which also restrain movement of hand 40 while within imaging system 20 such that the positioning of hand 40 is uniform or consistent from one imaging session to another imaging session and from one person to another person. In one implementation, imaging system 20 comprises structures that position and retain each individual finger of hand 40. In one implementation, imaging system 20 comprises a series of upwardly extending posts which guide positioning of the fingers, which separate the fingers and which restrain movement of the fingers.

As indicated by block 206 of FIG. 3, method 200 comprises identifying locations of a plurality of finger joints 42 of hand 40 based upon the first ultrasound images acquired with the transducer array in the first imaging mode. In one implementation, the first ultrasound images are of an entirety of hand 40. In yet another implementation, the first ultrasound images are of general areas of hand 40 which are predicted to encompass joints 42 and which are determined based upon the defined positioning of hand 40. For example, in one implementation, imaging system 20 may include hand fixtures or other structures which retain and 38 against movement in a predefined position and orientation. Based upon this predefined position and orientation of hand 40, as defined by the hand fixtures, controller 38 determines or identifies particular initial areas of hand 40 from which the first ultrasound images are to be acquired, wherein the initial areas of hand 40 are less than the entire area of hand 40, but may be greater than the subsequently defined and potentially more precise focus regions 48 which are based upon the subsequently acquired first ultrasound images. As indicated by block 206, method 200 then utilizes the first ultrasound images of such areas to more precisely locate each of finger joints 42.

In one implementation, first ultrasound images used to identify locations of finger joints 42 in block 206 comprise b-mode ultrasound images, resulting from the ultrasound imaging probe or transducer array 30 operating in a b-mode scan to generate the b-mode ultrasound images. Based upon the b-mode ultrasound images, the locations of joints 42 are identified.

As indicated by block 208 of FIG. 3, method 200 further comprises acquiring second ultrasound images of the plurality of finger joints 42 with the transducer array/probe 36 while the hand 40 is held stationary in the mechanical imaging system 20. The second ultrasound images are acquired using a second imaging mode that is different from the first imaging mode that was used to acquire the first ultrasound images. The second ultrasound images are of an area less than the entire area of hand 40 based upon the identified locations of finger joints 42 from block 206.

In one implementation, the second ultrasound images of the finger joints 42 are acquired while transducer array/probe 36 is operating in a Power Doppler Imaging (PDI) mode or high resolution PDI imaging mode. In one implementation, the second ultrasound images of the finger joints 42 are each acquired for a period of time that is at least as long as one cardiac cycle. As a result, any risk that the peak flow (representing the level of inflammation) will be missed is reduced. In addition, by acquiring PDI or high resolution PDI data over a period longer than a cardiac cycle, all the movie clips or images may be synchronized together to form a coherent cardiac cycle so that when you superimpose them over the optical image, the representation is realistic, and the peak flow (during systole) would appear together on all the joints. In one implementation, such synchronization is performed by modeling the cardiac cycle from the ultrasound information itself. This may be done by looking at the flow information (PDI or high-res PDI), and assigning the peak-systole point to the point of maximal flow. In other implementations, such resychronization is performed using an ECG device.

In one implementation, the second ultrasound images are acquired over a range of or for a focus region 48 having an area of 9 cm² for each of the plurality of identified finger joints 42. In one implementation, the second ultrasound images are acquired over range of or for a focus region 48 having a width of less than or equal to 3 cm for each of the plurality of identified finger joints 42.

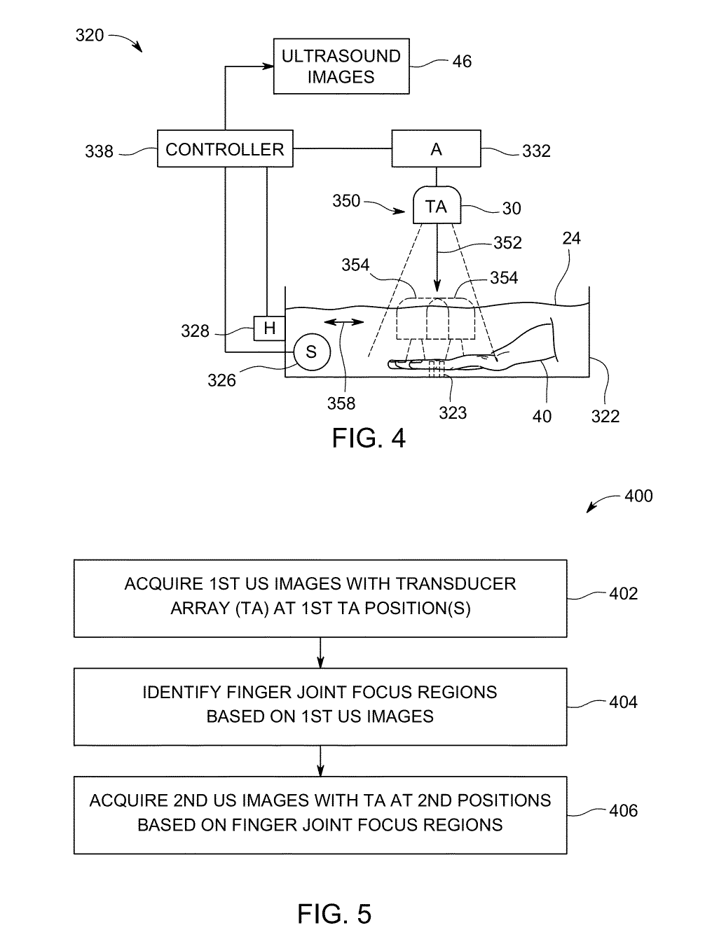

FIG. 4 schematically illustrates finger joint ultrasound imaging system 320, another example of finger joint ultrasound imaging system 20. Finger joint ultrasound imaging system 320 comprises fluid container 322, hand retainers 323, fluid 24, temperature sensors 326, heaters 328, transducer array 30, actuator 332 and controller 338. Fluid container 322 comprises a receptacle for containing fluid 24 (described above). Fluid container 322 is sized and configured to receive hand 40 such that hand 40 as immersed within fluid 24.

Hand retainers 323 comprise structures within container 322 which are shaped, sized and/or otherwise configured to retain hand 40 in a stationary state immersed within fluid 24 within container 322. In the example illustrated, hand retainers 323 additionally retain hand 40 in a predefined geo-referenced position or location within container 322. In the example illustrated, hand retainers 323 comprise posts projecting from a floor of container 322, wherein portions of hand 40 are received and retained between such posts.

Temperature sensors 326 comprise devices that sense the temperature fluid 24 within container 322. In one implementation, temperature sensors 326 submersed within fluid 24. In another implementation, temperature sensors 326 are external to container 324 to output signals indicating the temperature fluid 24 contained within container 322. Signals from sensor 326 are communicated to controller 328.

Heater 328 comprises a heating element or multiple heating elements located and operable to apply heat to fluid 24. In one implementation, heater 328 is located external to container 322, wherein he is conducted to fluid 24. In another implementation, heater 328 is submersed within fluid 24. In one implementation, heater 328 comprises an electric heater. In another implementation, heater 328 may comprise other types of heating elements. As will be described hereafter, controller 338 utilizes temperature sensors 326 and heater 328 to regulate the temperature fluid 24. In some implementations, sensor 326 and heater 328 are omitted.

Actuator 332 comprises a mechanism for selectively positioning transducer array 30 (described above) with respect to hand 40 submersed within fluid 24 within container 322 in response to control signals received from controller 338. In one implementation, actuator 332 is configured to selectively position transducer array 30 along each of three orthogonal axes. In one implementation, actuator 332 comprises an arrangement of arms or guide rails slidably or movably supporting transducer array 30 in one or more motors, such a stepper motors, which selectively move transducer array 30 along the arrangement of guide rails. For example, in one implementation, actuator 332 comprises a vertical arm horizontally driven along a first guide rail extending along a first horizontal axis by a first motor connected to the arm by a worm gear, threaded shaft or belt and a horizontal arm vertically driven along a second guide rail extending along a vertical axis by second motor connected to the horizontal arm by worm gear, threaded shaft or belt, wherein the transducer array is horizontally driven along a third guide rail extending along a second horizontal axis, perpendicular to the first horizontal axis, by a third motor connected to the transducer array 30 by a worm gear, threaded shaft or belt. In yet other implementations, actuator 332 may have other configurations.

Controller 338 comprises a processing unit that controls operation of heater 328, actuator 332 and transducer array 30. In the example illustrated, controller 338 outputs control signals based upon signals from after sensors 326 so as to control heater 328 and regulate the temperature fluid 24 for patient comfort and to facilitate imaging of joints 42 (shown in FIG. 1). In one implementation, controller 338 is regulated to a temperature to reduce inflammation and facilitate detection of very small, slow and shallow blood flow inside the finger joint when inflammation occurs as a result of rheumatoid arthritis. In one implementation, controller 338 maintains a temperature of fluid 24, when receiving hand 40, at a warm temperature of at least 20 degrees Celsius and nominally at least 37 degrees Celsius.

Controller 338 further controls the operation of transducer array 30 and actuator 332 to carry out method 400 shown in FIG. 5. As indicated by block 402 of FIG. 5, controller 338, following instructions or code contained in a non-transitory computer readable medium, outputs control signals causing actuator 332 to move transducer array 30 from an initial retracted, withdrawn position 350 in the direction indicated by arrow 352 to a one or more deployed sensing positions 354. In one implementation, such one or more sensing positions 354 are spaced vertically above hand 40 or the plane of hand 40 by less than or equal to 2 cm and nominally 1 cm. While transducer array 30 is at a sensing position, controller 338 outputs control signals causing transducer array 30 to acquire first joint locating ultrasound images. In one implementation, transducer array 30 is sized such that a first joint locating ultrasound image of an entirety of hand 40 is acquired while transducer array 30 is at a single position. In other implementations, controller 33 directs actuator 332 to move transducer array 30 between multiple sensing positions to acquire multiple first joint locating ultrasound images. In one implementation, controller 338 actuates transducer array 30 to a b-mode to acquire the first joint locating ultrasound images.

As indicated by block 404 of FIG. 5, once the first joint locating ultrasound images are acquired, controller 338, following programmed logic or a programmed algorithm, identifies finger joint focus regions 48 (shown and described above with respect to FIG. 1) based upon the first joint locating ultrasound images.

As indicated by block 406 of FIG. 5, controller 338 outputs control signals causing actuator 332 to move transducer array 30, in the direction of arrows 358 to second joint focused positions at or adjacent to each of the identified finger joint focus regions 48. While transducer array 30 is opposite to each identified finger joint focus region 48, controller 338 directs transducer assembly 30 to acquire second joint focused ultrasound images. In one implementation, controller 338 switches transducer array 30 to a different imaging mode as compared to the imaging mode by which the first joint locating images were acquired.

In one implementation, controller 338 switches transducer array 30 to a Power Doppler Imaging (PDI) mode or a high resolution PDI mode for acquisition of the second joint focused ultrasound images. In one implementation, for each joint 42 or for each focus region 48, controller 338 directs transducer array 30 to scan at least 20 and nominally at least 30 PDI or high-resolution PDI slices/scans. In one implementation, each slice has a thickness of 1 mm, resulting in a 2 to 3 cm wide area covering focus region 48. In some implementations, the thickness of each slice/scan may vary depending upon motor resolution, the resolution at which actuator 332 may reposition transducer array 30. In one implementation, for each joint 42, controller 338 directs transducer assembly 30 to acquire the second joint focused ultrasound images for a period of time that is at least as long as one cardiac cycle. In one implementation, each slice or scan occurs for a time period of at least two seconds.

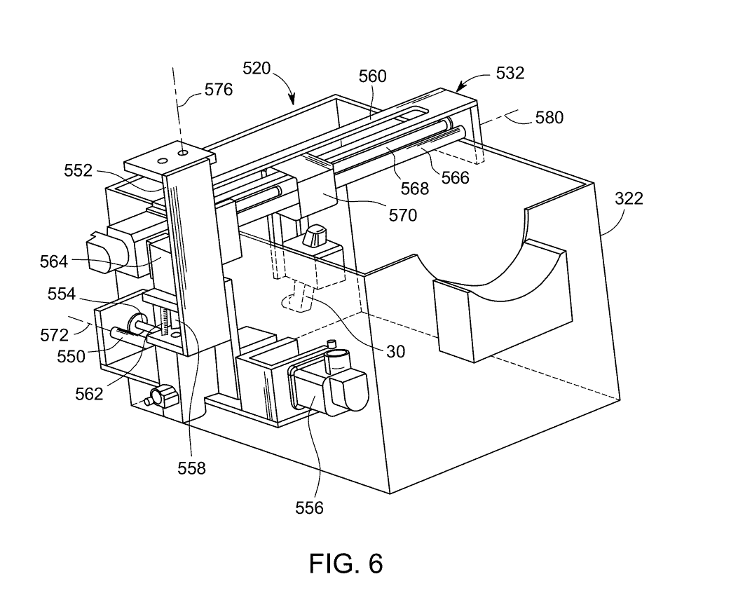

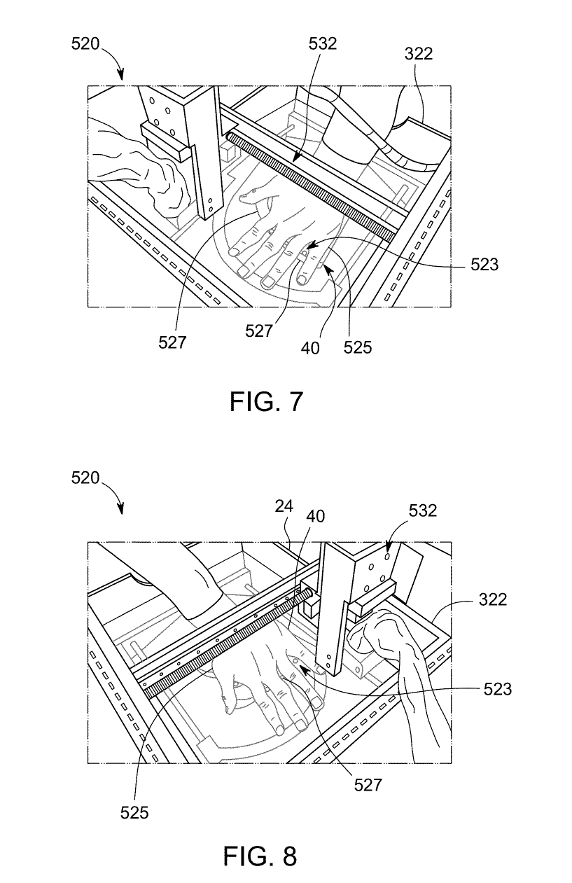

FIGS. 6-8 illustrate ultrasound finger joint imaging system 520, an example implementation of ultrasound finger joint imaging systems 20 and 320. Ultrasound finger joint imaging system 520 is similar to system 320 except that system 520 specifically illustrates hand retainer 523 (shown in FIGS. 7 and 8), an example of hand retainer 323, and actuator 532, an example of actuator 332. Those remaining components or elements of system 520 which correspond to components or elements of system 320 are numbered similarly or are shown in FIG. 4.

As shown by FIGS. 7 and 8, hand retainer 523 comprises a hand fixture which receives and 40 while hand 40 is submersed within liquid 24. Hand retainer 523 comprises a semicircular or U-shaped rear portion 525 and posts 527. Rear portion 525 receives a base of hand 40, just distal a person's wrist, with the fingers extending forwardly. In the example illustrated, rear portion 525 is rounded to conform to the round shape at the base of a person's hand. Rear portion extends forwardly to the base of each of the digits. Posts 527 are located so as to extend between each of the digits. In the example illustrated, the particular post 527 extending between the thumb and index finger is widened so through ergonomically contact sides of the person's palm and thumb. In other implementations, hand retainer 523 may have other configurations.

Actuator 532 comprise a movable support that facilitates selective repositioning of transducer array 30 with respect to hand 40 within container 322. In the example illustrated, actuator 532 comprises horizontal guide rail 550, vertical arm 552, threaded shaft 554, motor 556, vertical guide rail 558, horizontal arm 560, threaded shaft 562, motor 564, horizontal guide rail 566, threaded shaft 568 and motor 570. Horizontal guide rail 550 extends along axis 572 and movably supports vertical arm 552 for horizontal, sliding translation along guide rail 550. Threaded shaft 554 is connected to motor 556 and comprises external threads that engage internal threads connected to vertical arm 552. Motor 556, in response to signals from controller 338 (shown in FIG. 4), drives shaft 554 to horizontally translate vertical arm 552 along axis 572.

Vertical guide rail 558 extends along axis 576 and movably supports horizontal arm 560 for vertical movement along guide rail 558. Threaded shaft 562 is connected to motor 564 and comprises external threads that engage internal threads connected to horizontal arm 560. Motor 556, in response to signals from controller 338 (shown in FIG. 4), drives shaft 562 to vertically translate horizontal arm 560 along axis 576.

Horizontal guide rail 566 extends along axis 580 and movably supports transducer array 30 for horizontal movement along guide rail 566. Threaded shaft 568 is connected to motor 570 and comprises external threads that engage internal threads connected to transducer array 30. Motor 570, in response to signals from controller 338 (shown in FIG. 4), drives shaft 566 to horizontally translate transducer array 30 along axis 580. In other implementations, actuator 532 may have other configurations.

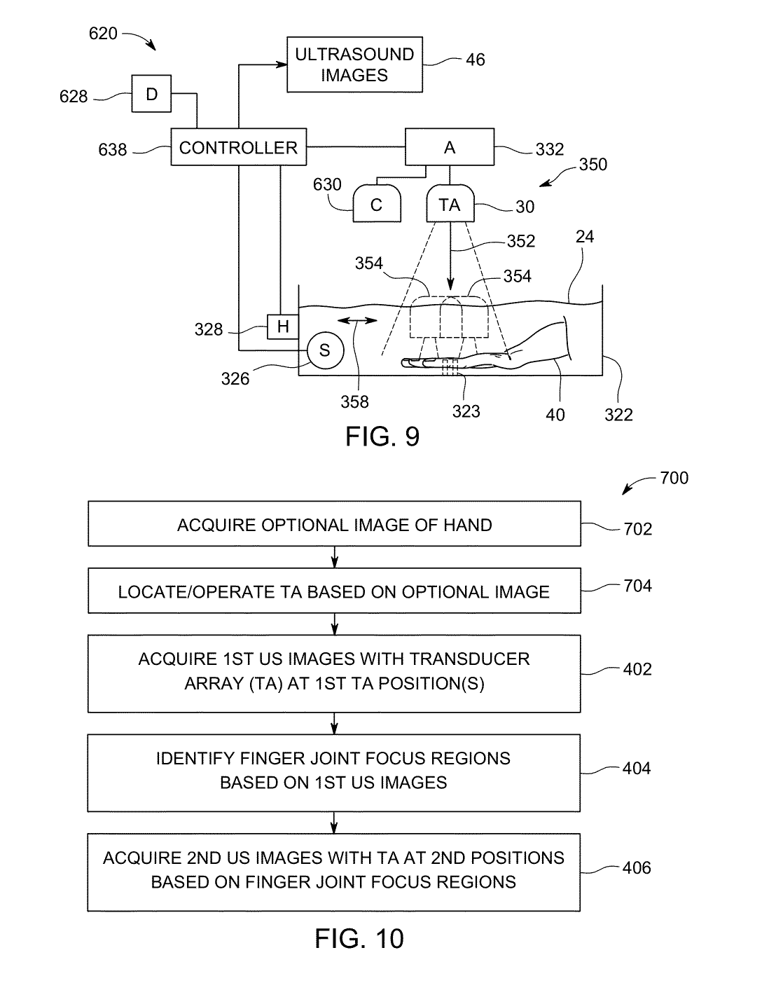

FIG. 9 schematically illustrates finger joint ultrasound imaging system 620, another example implementation of system 20 and system 520. Finger joint ultrasound imaging system 620 is similar to system 520 except that system 620 additionally comprises display 628, optical imaging device or camera 630 and controller 638. Those remaining components or elements of system 620 which correspond to components or elements of system 20 and/or system 320 are numbered similarly.

Display 628 comprises a display screen or monitor upon which images and/or data are visually presented. In one implementation display 628 is part of a console. In another implementation, display 628 is part of a portable electronic device, such as a smart phone or tablet. Display 628 communicates with controller 638 in a wired or wireless fashion. As will be described hereafter come display 628 presents images of a hand and/or finger joints of the hand based upon ultrasound images acquired by system 620.

Camera 630 comprises a digital or optical imaging device configured to capture and optical image of the hand, such as hand 40. In one implementation, camera 630 comprises an optical system that uses a lens and a variable diaphragm to focus light onto an electronic image pickup device, such as a CCD (charge coupled device) or CMOS (complementary metal-oxide semi conductor) sensor. In the example illustrated, camera 630 is operably coupled to actuator 332 such an actuator 332, operating under the control of signals from controller 338, repositions camera 630 to scan across hand 40. In other implementations, camera 630 is supported independent of actuator 332 and is stationary, wherein camera 630 captures and optical image of the entire hand 40 or relevant portions of hand 40 while stationary.

Controller 638 is similar to controller 38 and controller 338 except that controller 638 is configured to additionally operate in another mode using data or signals acquired from camera 630. Controller 638 comprises code, software, circuitry or other program logic is strictly process or to carry out method 700 outlined in FIG. 10. Method 700 is similar to method 400 except the method 700 additionally includes preliminary steps 702 and 704. Method 700 utilizes signals from camera 630 to perform a preliminary hand in joint identification step prior to carrying out ultrasound imaging in steps 402 and 406. As a result, acquiring ultrasound images of the finger joints is less time-consuming and more efficient.

As indicated by block 702 in FIG. 10, controller 638 outputs control signals to camera 630 to acquire and optical image of hand 40. In one implementation, acquiring an optical image of hand 40 comprises outputting control signals actuator 332 to selectively reposition camera 630 and capture multiple images at multiple locations, wherein the multiple images are aggregated to complete an image of hand 40. In another implementation, camera 630 stationary as a captures an image of hand 40. In one implementation, images are captured at various locations and/or angles to facilitate the creation of a three-dimensional image of hand 40.

As indicated by block 704 in FIG. 10, controller 638 utilizes the acquired optical image of hand 40 to locate and/or operate transducer array 30. In one implementation, controller 638 uses the optical image of hand 40, using digital image analysis, to identify proximate or general regions or locations for the finger joints. Controller 638 uses the identified general locations for the finger joints to generate control signals which cause actuator 332 to appropriately position transducer array 30 proximate to each of the identified general finger joint locations for the acquisition of the first ultrasound images and step 402. In addition or as an alternative, controller 638 further uses the identified general locations for the finger joints, derived from the acquired optical image of hand 40, to change one or more operational settings of transducer array 30 for the acquisition of the ultrasound images in step 402. For example, in one implementation, controller 638 reconstructs a three-dimensional image of hand 40, or a controller 638 utilizes a three-dimensional image to control actuator 332 to locate or position transducer array 30 in close proximity above the general finger joint regions of hand 40. The remaining steps of method 700, as indicated by block 402, 404 and 406 are described above.

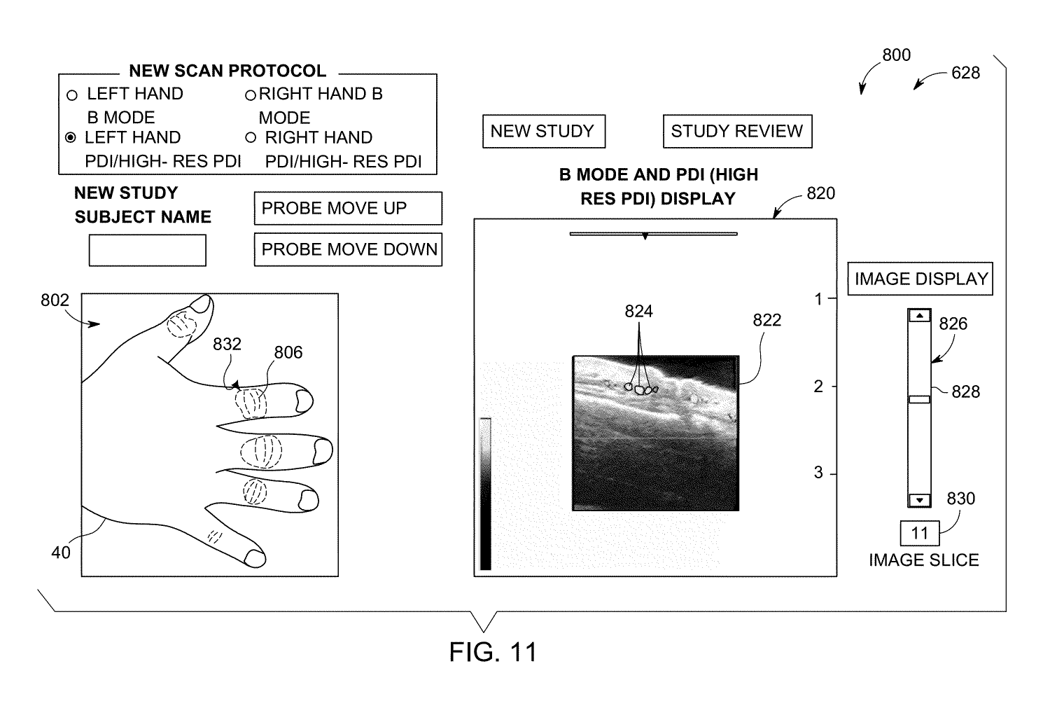

FIG. 11 illustrates one example selectable operational display mode for system 620. FIG. 11 illustrates an example screenshot 800 being presented on display 628 by controller 638. As shown by FIG. 11, controller 638 presents and acquired optical image 802 of hand 40 on display screen 638. In the example mode illustrated in FIG. 11, controller 638 additionally overlays or superimposes inflammation indicators 806 on the optical image 802 being presented, wherein the inflammation indicators 806 are located based upon the acquired ultrasound images of hand 40 indicating distribution of information in hand 40. As a result, the caretaker and/or patient may visibly ascertain where such information exists with respect to the actual optical image of hand 40.

In one implementation, inflammation indicators 806 comprise stippling, particles, specs, dots or other dispersed graphical point overlaid upon optical image 802 of hand 40 at locations where controller 638 has identified levels of inflammation exceeding a predefined threshold. Such inflammation indicators 806 are superimposed in an “X-ray like” fashion over the optical image 802 of hand 40. In another implementation, such inflammation indicators 806 comprise semi-transparent graphic superimposed upon the optical image 802 of hand 40 at locations where controller 638 has identified levels of inflammation exceeding a predefined threshold.

In one mode of operation, controller 638 additionally configures inflammation indicators 806 to identify not only the location of inflammation, but the extent or degrees of inflammation at the different locations of the finger joints of hand 40. In one implementation, controller 638 varies a density of the different regions of inflammation indicators 806 to indicate the degree of inflammation. For example, in implementations where inflammation indicators 806 comprise specks, dots or particles, controller 638 left in shown in FIG. 9) controls the density (number of specks, dots particles per unit area) based upon the level or degree of inflammation. In implementations where inflammation indicators 806 comprise semi-transparent regions or semi-transparent structures or graphics, controller 638 varies the opacity or translucency of the different regions based upon the level or degree of inflammation. In yet other implementations, controller 638 varies a shape of the individual particles, specks or dots to indicate different degrees of inflammation. In yet another implementation, controller 638 varies a grayscale, brightness, shade and/or color of the particles, specks or dots or of the semi-transparent region or structures to indicate the level or degree of inflammation superimposed upon the optical image 802 of hand 40.

In the example illustrated in FIG. 11, system 620 presents slices are sectional views of hand 40, illustrating inflammation within hand 40. In the example shown in FIG. 11, controller 638 presents image slice 820 of a finger joint 822 with inflammation 824. In the example illustrated, controller 638 presents a graphical user interface 826 having a slider bar 828 which may be raised and lowered (with a mouse, pointer or by finger touch) to relocate or reposition the image plane for the slice of the finger joint being presented. As indicated in box 830, the finger slices are numbered or otherwise distinguished from one another.

In one mode of operation, optical image 802 presented on display 628 is utilized by controller 638 as a graphical user interface, allowing a caretaker or user to easily select specific finger joints or portions of hand 40 for more detailed analysis, such as for being presented as an ultrasound slice, such as slice 820. In particular, each location on optical image 802 is selectable such as by locating a cursor 832 on top of a portion of the optical image 802 and selecting the portion or specific joint of the optical image 802. Such selections may alternatively be carried out by manual touch inputs of a user when display 628 is a touch screen. Once a particular finger joint has been selected on optical image 802, an enlarged view of the selected finger joint is shown on display 628. Alternatively, in another mode of operation, an ultrasound slice, such as slice 820, of the selected finger joint is presented on display 628. As noted above, graphical user interface 826 allows the user to relocate the image slice within the selected finger joint.

While the preferred embodiments of the invention have been illustrated and described, it will be appreciated that various changes can be made therein without departing from the spirit and scope of the invention. For example, although different example embodiments may have been described as including one or more features providing one or more benefits, it is contemplated that the described features may be interchanged with one another or alternatively be combined with one another in the described example embodiments or in other alternative embodiments. One of skill in the art will understand that the invention may also be practiced without many of the details described above. Accordingly, it will be intended to include all such alternatives, modifications and variations set forth within the spirit and scope of the appended claims. Further, some well-known structures or functions may not be shown or described in detail because such structures or functions would be known to one skilled in the art. Unless a term is specifically and overtly defined in this specification, the terminology used in the present specification is intended to be interpreted in its broadest reasonable manner, even though may be used conjunction with the description of certain specific embodiments of the present invention.

What is claimed is:

1. A method of acquiring ultrasound images for a hand examination, the method comprising:
receiving a hand in a scanning assembly including a transducer array and a fluid providing an acoustic coupling between the transducer array and the hand;
identifying locations of a plurality of finger joints of the hand while the hand is held stationary in the scanning assembly;
acquiring ultrasound images of the plurality of finger joints with the transducer array while the hand is held stationary in the scanning assembly, the ultrasound images being of an area less than an entire area of the hand based on the identified locations of the finger joints of the hand.

2. The method of claim 1, wherein the transducer array is mounted to a movable support.

3. The method of claim 1, further comprising displaying at least one of the images of the finger joints.

4. The method of claim 1, wherein the ultrasound images comprise Power Doppler Imaging (PDI) images or High Res PDI images.

5. The method of claim 1, wherein identifying the locations of the plurality of finger joints comprises manually identifying the locations of the plurality of finger joints.

6. The method of claim 1, further comprising sensing a position of the hand, and wherein identifying locations of the plurality of finger joints comprises automatically identifying the locations of the plurality of finger joints based on the sensed position of the hand.

7. The method of claim 1, wherein identifying the locations of the plurality of finger joints comprises acquiring a b-mode ultrasound image of the hand and automatically identifying the positions of the plurality of finger joints based on the b-mode ultrasound image.

8. The method of claim 1 further comprising receiving signals representing an optical image of the hand, wherein identifying the locations of the plurality of finger joints comprises digitally analyzing the optical image.

9. The method of claim 1 further comprising:
reconstructing a three-dimensional image of the hand; and
automatically adjusting positioning of the transducer array with respect to the hand based upon the three-dimensional image of the hand.

10. The method of claim 1 further comprising:
presenting an image of at least a portion of the hand; and
superimposing a representation of inflammation on the image based upon the acquired ultrasound images.

11. The method of claim 1, wherein the transducer array is large enough to scan the whole hand without moving the transducer array.

12. The method of claim 1, wherein the scanning assembly comprises a moveable support connected to the transducer array and an actuator connected to the transducer array, and further comprising controlling the actuator to automatically position the transducer array based on the identified locations of the finger joints.

13. The method of claim 12, wherein acquiring ultrasound images of the plurality of finger joints comprises capturing ultrasound images over a range of less than or equal to 3 cm for each identified finger joint.

14. The method of claim 1, further comprising controlling the temperature of the fluid with a heater at least one of before said acquiring the ultrasound images and after said acquiring the ultrasound images.

15. A method of acquiring ultrasound images for a rheumatoid arthritis examination, the method comprising:
receiving a hand in a mechanical scanning assembly, the mechanical scanning assembly comprising a transducer array and a fluid providing an acoustic coupling between the transducer array and the hand;
acquiring first ultrasound images of the hand with the transducer array in a first imaging mode while the hand is held stationary in the mechanical scanning assembly;
identifying locations of a plurality of finger joints of the hand based on the first ultrasound images;
acquiring second ultrasound images of the plurality of finger joints with the transducer array while the hand is held stationary in the mechanical scanning assembly, the second ultrasound images acquired using a second imaging mode that is different from the first imaging mode, and the second ultrasound images being of an area less than an entire area of the hand based on the identified locations of the finger joints of the hand.

16. The method of claim 15, wherein the first ultrasound images comprise B-mode images.

17. The method of claim 16, wherein the second ultrasound images comprise power Doppler imaging (PDI) images or High Res PDI images.

18. The method of claim 16, wherein the first ultrasound images are acquired of the entire hand.

19. The method of claim 16, wherein the first ultrasound images are acquired of less than the entire hand.

20. The method of claim 15, wherein identifying the locations of the plurality of finger joints comprises automatically identifying the locations using an image processing technique.

21. The method of claim 15 further comprising receiving signals representing an optical image of the hand, wherein identifying the locations of the plurality of finger joints comprises digitally analyzing the optical image.

22. The method of claim 15 further comprising:
reconstructing a three-dimensional image of the hand; and
automatically adjusting positioning of the transducer array with respect to the hand based upon the three-dimensional image of the hand.

23. The method of claim 15 further comprising:
presenting an image of at least a portion of the hand; and
superimposing a representation of inflammation on the image based upon the acquired ultrasound images.

24. The method of claim 15 comprising:
presenting an optical image of the hand;
using the optical image as a graphical user interface whereby individual finger joints are selectable for further image presentation on a display.

25. The method of claim 15, wherein the hand is held stationary with a fixture or a localizing structure.

26. The method of claim 15, further comprising controlling an actuator to position the transducer array within 2 cm of the hand while acquiring the first ultrasound images.

27. The method of claim 15, further comprising automatically controlling the actuator to move the transducer array in order to acquire the second ultrasound images.

28. The method of claim 15, wherein acquiring the second ultrasound images comprises acquiring the ultrasound images over a range of less than 3 cm for each of the plurality of identified finger joints.

29. The method of claim 15, wherein acquiring the second plurality of ultrasound images comprises acquiring each of the second plurality of ultrasound images for a period of time that is at least as long as one cardiac cycle.
